SEQUENCE LISTING (26 sequences)

SEQ ID NO: 1 - protein, 3011 aa, Hepatitis C virus
MSTNPKPQRKTKRNTNRRPQDVKFPGGGQIVGGVYLLPRRGPRLGVRATRKTSERSQPRGRRQPIPKARRPEGRTWAQPGYPWPLYGNEGCGWAGWLLSPRGSRPSWGPTDPRRRSRNLGKVIDTLTCGFADLMGYIPLVGAPLGGAARALAHGVRVLEDGVNYATGNLPGCSFSIFLLALLSCLTVPASAYQVRNSSGLYHVTNDCPNSSIVYEAADAILHTPGCVPCVREGNASRCWVAVTPTVATRDGKLPTTQLRRHIDLLVGSATLCSALYVGDLCGSVFLVGQLFTFSPRRHWTTQDCNCSIYPGHITGHRMAWDMMMNWSPTAALVVAQLLRIPQAIMDMIAGAHWGVLAGIAYFSMVGNWAKVLVVLLLFAGVDAETHVTGGSAGRTTAGLVGLLTPGAKQNIQLINTNGSWHINSTALNCNESLNTGWLAGLFYQHKFNSSGCPERLASCRRLTDFAQGWGPISYANGSGLDERPYCWHYPPRPCGIVPAKSVCGPVYCFTPSPVVVGTTDRSGAPTYSWGANDTDVFVLNNTRPPLGNWFGCTWMNSTGFTKVCGAPPCVIGGVGNNTLLCPTDCFRKHPEATYSRCGSGPWITPRCMVDYPYRLWHYPCTINYTIFKVRMYVGGVEHRLEAACNWTRGERCDLEDRDRSELSPLLLSTTQWQVLPCSFTTLPALSTGLIHLHQNIVDVQYLYGVGSSIASWAIKWEYVVLLFLLLADARVCSCLWMMLLISQAEAALENLVILNAASLAGTHGLVSFLVFFCFAWYLKGRWVPGAVYAFYGMWPLLLLLLALPQRAYALDTEVAASCGGVVLVGLMALTLSPYYKRYISWCMWWLQYFLTRVEAQLHVWVPPLNVRGGRDAVILLMCVVHPTLVFDITKLLLAIFGPLWILQASLLKVPYFVRVQGLLRICALARKIAGGHYVQMAIIKLGALTGTYVYNHLTPLRDWAHNGLRDLAVAVEPVVFSRMETKLITWGADTAACGDIINGLPVSARRGQEILLGPADGMVSKGWRLLAPITAYAQQTRGLLGCIITSLTGRDKNQVEGEVQIVSTATQTFLATCINGVCWTVYHGAGTRTIASPKGPVIQMYTNVDQDLVGWPAPQGSRSLTPCTCGSSDLYLVTRHADVIPVRRRGDSRGSLLSPRPISYLKGSSGGPLLCPAGHAVGLFRAAVCTRGVAKAVDFIPVENLETTMRSPVFTDNSSPPAVPQSFQVAHLHAPTGSGKSTKVPAAYAAQGYKVLVLNPSVAATLGFGAYMSKAHGVDPNIRTGVRTITTGSPITYSTYGKFLADGGCSGGAYDIIICDECHSTDATSILGIGTVLDQAETAGARLVVLATATPPGSVTVSHPNIEEVALSTTGEIPFYGKAIPLEVIKGGRHLIFCHSKKKCDELAAKLVALGINAVAYYRGLDVSVIPTSGDVVVVSTDALMTGFTGDFDSVIDCNTCVTQTVDFSLDPTFTIETTTLPQDAVSRTQRRGRTGRGKPGIYRFVAPGERPSGMFDSSVLCECYDAGCAWYELTPAETTVRLRAYMNTPGLPVCQDHLEFWEGVFTGLTHIDAHFLSQTKQSGENFPYLVAYQATVCARAQAPPPSWDQMWKCLIRLKPTLHGPTPLLYRLGAVQNEVTLTHPITKYIMTCMSADLEVVTSTWVLVGGVLAALAAYCLSTGCVVIVGRIVLSGKPAIIPDREVLYQEFDEMEECSQHLPYIEQGMMLAEQFKQKALGLLQTASRQAEVITPAVQTNWQKLEVFWAKHMWNFISGIQYLAGLSTLPGNPAIASLMAFTAAVTSPLTTGQTLLFNILGGWVAAQLAAPGAATAFVGAGLAGAAIGSVGLGKVLVDILAGYGAGVAGALVAFKIMSGEVPSTEDLVNLLPAILSPGALVVGVVCAAILRRHVGPGEGAVQWMNRLIAFASRGNHVSPTHYVPESDAAARVTAILSSLTVTQLLRRLHQWISSECTTPCSGSWLRDIWDWICEVLSDFKTWLKAKLMPQLPGIPFVSCQRGYRGVWRGDGIMHTRCHCGAEITGHVKNGTMRIVGPRTCRNMWSGTFPINAYTTGPCTPLPAPNYKFALWRVSAEEYVEIRRVGDFHYVSGMTTDNLKCPCQIPSPEFFTELDGVRLHRFAPPCKPLLREEVSFRVGLHEYPVGSQLPCEPEPDVAVLTSMLTDPSHITAEAAGRRLARGSPPSMASSSASQLSAPSLKATCTANHDSPDAELIEANLLWRQEMGGNITRVESENKVVILDSFDPLVAEEDEREVSVPAEILRKSRRFARALPVWARPDYNPPLVETWKKPDYEPPVVHGCPLPPPRSPPVPPPRKKRTVVLTESTLSTALAELATKSFGSSSTSGITGDNTTTSSEPAPSGCPPDSDVESYSSMPPLEGEPGDPDLSDGSWSTVSSGADTEDVVCCSMSYSWTGALVTPCAAEEQKLPINALSNSLLRHHNLVYSTTSRSACQRQKKVTFDRLQVLDSHYQDVLKEVKAAASKVKANLLSVEEACSLTPPHSAKSKFGYGAKDVRCHARKAVAHINSVWKDLLEDSVTPIDTTIMAKNEVFCVQPEKGGRKPARLIVFPDLGVRVCEKMALYDVVSKLPLAVMGSSYGFQYSPGQRVEFLVQAWKSKKTPMGFSYDTRCFDSTVTESDIRTEEAIYQCCDLDPQARVAIKSLTERLYVGGPLTNSRGENCGYRRCRASGVLTTSCGNTLTCYIKARAACRAAGLQDCTMLVCGDDLVVICESAGVQEDAASLRAFTEAMTRYSAPPGDPPQPEYDLELITSCSSNVSVAHDGAGKRVYYLTRDPTTPLARAAWETARHTPVNSWLGNIIMFAPTLWARMILMTHFFSVLIARDQLEQALNCEIYGACYSIEPLDLPPIIQRLHGLSAFSLHSYSPGEINRVAACLRKLGVPPLRAWRHRARSVRARLLSRGGRAAICGKYLFNWAVRTKLKLTPIAAAGRLDLSGWFTAGYSGGDIYHSVSHARPRWFWFCLLLLAAGVGIYLLPNR

SEQ ID NO: 2 - protein, 3010 aa, Hepatitis C virus
MSTNPKPQRKTKRNTNRRPQDVKFPGGGQIVGGVYLLPRRGPRLGVRATRKTSERSQPRGRRQPIPKARQPEGRAWAQPGYPWPLYGNEGLGWAGWLLSPRGSRPSWGPTDPRRRSRNLGKVIDTLTCGFADLMGYIPLVGAPLGGAARALAHGVRVLEDGVNYATGNLPGCSFSIFLLALLSCLTIPASAYEVRNVSGVYHVTNDCSNASIVYEAADMIMHTPGCVPCVRENNSSRCWVALTPTLAARNASVPTTTIRRHVDLLVGAAALCSAMYVGDLCGSVFLVAQLFTFSPRRHETVQDCNCSIYPGHVTGHRMAWDMMMNWSPTAALVVSQLLRIPQAVVDMVAGAHWGVLAGLAYYSMVGNWAKVLIVMLLFAGVDGGTYVTGGTMAKNTLGITSLFSPGSSQKIQLVNTNGSWHINRTALNCNDSLNTGFLAALFYVHKFNSSGCPERMASCSPIDAFAQGWGPITYNESHSSDQRPYCWHYAPRPCGIVPAAQVCGPVYCFTPSPVVVGTTDRFGVPTYSWGENETDVLLLNNTRPPQGNWFGCTWMNSTGFTKTCGGPPCNIGGIGNKTLTCPTDCFRKHPEATYTKCGSGPWLTPRCLVHYPYRLWHYPCTVNFTIFKVRMYVGGVEHRLEAACNWTRGERCNLEDRDRSELSPLLLSTTEWQVLPCSFTTLPALSTGLIHLHQNVVDVQYLYGIGSAVVSFAIKWEYVLLLFLLLADARVCACLWMMLLIAQAEAALENLVVLNAASVAGAHGILSFLVFFCAAWYIKGRLVPGAAYALYGVWPLLLLLLALPPRAYAMDREMAASCGGAVFVGLILLTLSPHYKLFLARLIWWLQYFITRAEAHLQVWIPPLNVRGGRDAVILLTCAIHPELIFTITKILLAILGPLMVLQAGITKVPYFVRAHGLIRACMLVRKVAGGHYVQMALMKLAALTGTYVYDHLTPLRDWAHAGLRDLAVAVEPVVFSDMETKVITWGADTAACGDIILGLPVSARRGREIHLGPADSLEGQGWRLLAPITAYSQQTRGLLGCIITSLTGRDRNQVEGEVQVVSTATQSFLATCVNGVCWTVYHGAGSKTLAGPKGPITQMYTNVDQDLVGWQAPPGARSLTPCTCGSSDLYLVTRHADVIPVRRRGDSRGSLLSPRPVSYLKGSSGGPLLCPSGHAVGIFRAAVCTRGVAKAVDFVPVESMETTMRSPVFTDNSSPPAVPQTFQVAHLHAPTGSGKSTKVPAAYAAQGYKVLVLNPSVAATLGFGAYMSKAHGIDPNIRTGVRTITTGAPITYSTYGKFLADGGCSGGAYDIIICDECHSTDSTTILGIGTVLDQAETAGARLVVLATATPPGSVTVPHPNIEEVALSSTGEIPFYGKAIPIETIKGGRHLIFCHSKKKCDELAAKLSGLGLNAVAYYRGLDVSVIPTSGDVIVVATDALMTGFTGDFDSVIDCNTCVTQTVDFSLDPTFTIETTTVPQDAVSRSQRRGRTGRGRMGIYRFVTPGERPSGMFDSSVLCECYDAGCAWYELTPAETSVRLRAYLNTPGLPVCQDHLEFWESVFTGLTHIDAHFLSQTKQAGDNFPYLVAYQATVCARAQAPPPSWDQMWKCLIRLKPTLHGPTPLLYRLGAVQNEVTTTHPITKYIMACMSADLEVVTSTWVLVGGVLAALAAYCLTTGSVVIVGRIILSGKPAIIPDREVLYREFDEMEECASHLPYIEQGMQLAEQFKQKAIGLLQTATKQAEAAAPVVESKWRTLEAFWAKHMWNFISGIQYLAGLSTLPGNPAIASLMAFTASITSPLTTQHTLLFNILGGWVAAQLAPPSAASAFVGAGIAGAAVGSIGLGKVLVDILAGYGAGVAGALVAFKVMSGEMPSTEDLVNLLPAILSPGALVVGVVCAAILRRHVGPGEGAVQWMNRLIAFASRGNHVSPTHYVPESDAAARVTQILSSLTITQLLKRLHQWINEDCSTPCSGSWLRDVWDWICTVLTDFKTWLQSKLLPRLPGVPFFSCQRGYKGVWRGDGIMQTTCPCGAQITGHVKNGSMRIVGPRTCSNTWHGTFPINAYTTGPCTPSPAPNYSRALWRVAAEEYVEVTRVGDFHYVTGMTTDNVKCPCQVPAPEFFTEVDGVRLHRYAPACKPLLREEVTFLVGLNQYLVGSQLPCEPEPDVAVLTSMLTDPSHITAETAKRRLARGSPPSLASSSASQLSAPSLKATCTTRHDSPDADLIEANLLWRQEMGGNITRVESENKVVILDSFEPLQAEEDEREVSVPAEILRRSRKFPRAMPIWARPDYNPPLLESWKDPDYVPPVVHGCPLPPAKAPPIPPPRRKRTVVLSESTVSSALAELATKTFGSSESSAVDSGTATASPDQPSDDGDAGSDVESYSSMPPLEGEPGDPDLSDGSWSTVSEEASEDVVCCSMSYTWTGALITPCAAEETKLPINALSNSLLRHHNLVYATTSRSASLRQKKVTFDRLQVLDDHYRDVLKEMKAKASTVKAKLLSVEEACKLTPPHSARSKFGYGAKDVRNLSSKAVNHIRSVWKDLLEDTETPIDTTIMAKNEVFCVQPEKGGRKPARLIVFPDLGVRVCEKMALYDVVSTLPQAVMGSSYGFQYSPGQRVEFLVNAWKAKKCPMGFAYDTRCFDSTVTENDIRVEESIYQCCDLAPEARQAIRSLTERLYIGGPLTNSKGQNCGYRRCRASGVLTTSCGNTLTCYLKAAAACRAAKLQDCTMLVCGDDLVVICESAGTQEDEASLRAFTEAMTRYSAPPGDPPKPEYDLELITSCSSNVSVAHDASGKRVYYLTRDPTTPLARAAWETARHTPVNSWLGNIIMYAPTLWARMILMTHFFSILLAQEQLEKALDCQIYGACYSIEPLDLPQIIQRLHGLSAFSLHSYSPGEINRVASCLRKLGVPPLRVWRHRARSVRARLLSQGGRAATCGKYLFNWAVRTKLKLTPIPAASQLDLSSWFVAGYSGGDIYHSLSRARPRWFMWCLLLLSVGVGIYLLPNR

SEQ ID NO: 3 - protein, 447 aa, Hepatitis C virus
SGSWLRDIWDWICEVLSDFKTWLKAKLMPQLPGIPFVSCQRGYRGVWRGDGIMHTRCHCGAEITGHVKNGTMRIVGPRTCRNMWSGTFPINAYTTGPCTPLPAPNYKFALWRVSAEEYVEIRRVGDFHYVSGMTTDNLKCPCQIPSPEFFTELDGVRLHRFAPPCKPLLREEVSFRVGLHEYPVGSQLPCEPEPDVAVLTSMLTDPSHITAEAAGRRLARGSPPSMASSSASQLSAPSLKATCTANHDSPDAELIEANLLWRQEMGGNITRVESENKVVILDSFDPLVAEEDEREVSVPAEILRKSRRFARALPVWARPDYNPPLVETWKKPDYEPPVVHGCPLPPPRSPPVPPPRKKRTVVLTESTLSTALAELATKSFGSSSTSGITGDNTTTSSEPAPSGCPPDSDVESYSSMPPLEGEPGDPDLSDGSWSTVSSGADTEDVVC

SEQ ID NO: 4 - protein, 66 aa, Hepatitis C virus
KSRRFARALPVWARPDYNPPLVETWKKPDYEPPVVHGCPLPPPRSPPVPPPRKKRTVVLTESTLST

SEQ ID NO: 5 - protein, 66 aa, Hepatitis C virus
KSRKFPRAMPIWARPDYNPPLLESWKDPDYVPPVVHGCPLPPAKAPPIPPPRRKRTVVLSESTVSS

SEQ ID NO: 6 - protein, 24 aa, Hepatitis C virus
  misc_feature (6)..(6): Xaa can be any naturally occurring amino acid
KSRRFXRALPVWAXPXXXPPLVEX

SEQ ID NO: 7 - protein, 24 aa, Hepatitis C virus
KSRRFPRALPVWARPDYNPPLVEP

SEQ ID NO: 8 - DNA, 69 bp, Artificial Sequence
  oligonucleotide primer
ttcgctcgagccctgcccgtttgggcgcggccggactacaaccccccgctagtagagccctggaaaaag

SEQ ID NO: 9 - DNA, 25 bp, Artificial Sequence
  oligonucleotide primer
ccatgtatacgacattgagcagcag

SEQ ID NO: 10 - protein, 15 aa, Hepatitis C virus
LRRSRKFPRAMPIWA

SEQ ID NO: 11 - protein, 24 aa, Hepatitis C virus
  misc_feature (6)..(6): Xaa can be any naturally occurring amino acid
KSRRFXRALPVWARPDYNPPLVEX

SEQ ID NO: 12 - protein, 11 aa, Hepatitis C virus
  misc_feature (6)..(6): Xaa can be any naturally occurring amino acid
KSRRFXRALPV

SEQ ID NO: 13 - protein, 11 aa, Hepatitis C virus
KSRRFPRALPV

SEQ ID NO: 14 - protein, 9 aa, Hepatitis C virus
WARPDYNPP

SEQ ID NO: 15 - protein, 10 aa, Hepatitis C virus
RKKRTVVLTE

SEQ ID NO: 16 - protein, 7 aa, Hepatitis C virus
TEDVVCC

SEQ ID NO: 17 - protein, 11 aa, Hepatitis C virus
FRRPLPAWARP

SEQ ID NO: 18 - protein, 9 aa, Hepatitis C virus
EEDDTTVCC

SEQ ID NO: 19 - protein, 12 aa, Hepatitis C virus
RALPIWARPDYN

SEQ ID NO: 20 - protein, 10 aa, Hepatitis C virus
VSGSEDVVCC

SEQ ID NO: 21 - DNA, 45 bp, Artificial Sequence
  oligonucleotide primer
gtaattttggactctttcgatccgctcgtggcggaggaggatgag

SEQ ID NO: 22 - DNA, 25 bp, Artificial Sequence
  oligonucleotide primer
ccatgtatacgacattgagcagcag

SEQ ID NO: 23 - DNA, 54 bp, Artificial Sequence
  oligonucleotide primer
gagatcctgcggaagtccaggagattcgcccgagcgctgcccgtttgggcacgc

SEQ ID NO: 24 - DNA, 25 bp, Artificial Sequence
  oligonucleotide primer
ccatgtatacgacattgagcagcag

SEQ ID NO: 25 - DNA, 35 bp, Artificial Sequence
  oligonucleotide primer
gtttcgtcgacccttaccggcttgggcacggcctg

SEQ ID NO: 26 - DNA, 29 bp, Artificial Sequence
  oligonucleotide primer
ccaggtatacgacatggagcagcacacgg

(19) United States
(12) Patent Application Publication
(10) Pub. No.: US 2014/0271726 A1
(43) Pub. Date: Sep. 18, 2014

(54) COMPOSITIONS AND METHODS FOR PREDICTING HCV SUSCEPTIBILITY TO ANTIVIRAL AGENTS

(71) Applicant: WISCONSIN ALUMNI RESEARCH FOUNDATION, Madison, WI (US)

(72) Inventors: Israr H. Ansari, Middleton, WI (US); Robert T. Striker, Madison, WI (US)

(73) Assignee: WISCONSIN ALUMNI RESEARCH FOUNDATION, Madison, WI (US)

(21) Appl. No.: 13/796,974

(22) Filed: Mar. 12, 2013

(51) Int. Cl. C12Q 1/70; C07K 14/18

(52) U.S. Cl. CPC C12Q 1/70; C07K 14/1833
USPC 424/278.1; 536/23.72; 435/5; 506/17

(57) ABSTRACT

Provided herein are methods for determining the susceptibility of a hepatitis C virus (HCV) in a patient to anti-viral agents, particularly cyclophilin inhibitors such as cyclosporine A. More particularly, provided herein are methods for determining the amino acid sequence within a region of the HCV NS5A protein and comparing the viral amino acid sequence to that of a reference strain, wherein the existence of at least one variation in the viral genome is indicative that the virus is more or less susceptible to anti-viral agents relative to the reference strain. Also provided are isolated polynucleotide molecules, replicons, and kits that can be used to assay the susceptibility of a particular HCV to an anti-viral agent.

### CROSS-REFERENCE TO RELATED APPLICATION

Not Applicable.

### STATEMENT REGARDING FEDERALLY FUNDED RESEARCH OR DEVELOPMENT

Not Applicable.

### FIELD OF THE INVENTION

This invention relates generally to methods and compositions for customizing anti-viral medication treatment regimes for patients infected with hepatitis C virus (HCV). In particular, the invention is directed to methods and compositions that facilitate genetic comparisons between certain regions of a given HCV strain and a known consensus sequence to determine the susceptibility of the HCV strain to treatment with certain anti-viral agents.

### BACKGROUND OF THE INVENTION

Hepatitis C virus (HCV) was first characterized in 1989 (Choo et al., Science 244: 359-362, 1989), although its existence had been suspected for many years as the elusive cause of a liver disease referred to as non A-non B hepatitis (“NANBH”), with flu-like symptoms and occurring in many patients years after they receive blood transfusion. HCV is a single-stranded, plus-sense RNA virus of Flaviviridae, which includes viruses that cause bovine diarrhea, hog cholera, yellow fever, and tick-borne encephalitis. The HCV genome is approximately 9.5 kb in size, and is characterized by a unique open reading frame encoding a single poly-protein.

It is estimated that HCV infects about 170 million people worldwide, more than four times those infected with human immunodeficiency virus (“HIV”), and the number of HCV associated deaths may eventually overtake deaths caused by AIDS (Cohen, Science 285:26-30, 1999). The Center for Disease Control (CDC) has calculated that 1.8 percent of the U.S. population may be infected with HCV.

HCV infection is now known to be the leading cause of liver failure in the United States. Approximately 60% of HCV patients develop chronic liver disease and a substantial number of these patients have to undergo liver transplant. Unfortunately, the virus survives in other cells and eventually infects the new liver upon transplant. HCV infected patients have a higher mortality rate than non-HCV infected liver transplant patients at five years, likely due, at least in part, to accelerated HCV infection of the transplanted liver, leading to the recurrence of liver failure.

Immunosuppressive agents, or immunosuppressants, are invariably required for all allografts to blunt the recipient's immune response and minimize rejection. Use of immunosuppressants, however, has been linked to the increase in HCV virulence and in patient morbidity and mortality. This effect is especially pronounced in liver transplantation and is observed to a lesser extent in kidney transplantation.

Contradicting observations, however, have been widely reported with regard to some of the immunosuppressants, especially cyclosporine A (CsA). In some instances, CsA treatments are known to lead to an increase in virulence of HCV in the liver (see e.g., Everson, Impact of immunosuppressive therapy on recurrence of hepatitis C, Liver Transpl., 8(Suppl 1):S19-27, 2010), yet in other instances, CsA has been shown to inhibit HCV replication in vitro and has been used as a treatment for HCV infection. For example, Nakagawa et al. (Specific inhibition of hepatitis C virus replication by cyclosporin A, Biochem Biophys Res Commun 313(1):42-7, 2004) and Watashi et al. (Cyclosporin A suppresses replication of hepatitis C virus genome in cultured hepatocytes, Hepatology 38(5):1282-8, 2003) reported that CsA can inhibit HCV replication in vitro through a mechanism apparently unrelated to its immunosuppressive properties. Though CsA does not appear to control HCV effectively in liver transplant recipients, presumably due to its immunosuppressive effects, a study in Japan found that a six-month course of HCV treatment with a combination of CsA and alpha interferon was more effective at achieving sustained virological responses than interferon alone (42/76 [55%] vs. 14/44 [32%]; p=0.01) (Inoue et al., Combined interferon alpha2b and cyclosporin A in the treatment of chronic hepatitis C: controlled trial, J. Gastroenterol 38:567-72, 2003). Further research is focused on NIM811, Debio-025, SCY325, and various CsA analogs with varying degrees of immunosuppressive activity.

The inconsistency among the various reported research likely involves differences in study design, varying complexity of the patient population, such as differences in how patients respond to immunosuppressants, and other factors. The most likely cause of the inconsistency, however, is the high genetic heterogeneity of the HCV virus. Based upon phylogenetic analysis of the core, EI, and NS5 regions of the viral genome, the HCV virus has been classified into at least six genotypes and more than 30 subtypes dispersed throughout the world (Major and Feinstone, Hepatology 25:1527-1538, 1997; Clarke, J. Genl. Virol 78:2397-2410, 1997). It is believed that various genotypes or subtypes of HCV may be susceptible to inhibition by immunosuppressants such as cyclosporine A (CsA), while others may not. However, direct or specific correlation between the genotype of an HCV strain and its susceptibility to immunosuppressant treatment is lacking. As a consequence, the current practice it to modify CsA treatment of HCV in transplant patients in a reactionary manner based on viral load or increased virulence as evidenced by tissue destruction—these being the only indicators of failure of CsA treatment of HCV.

There is, therefore, a need to determine the susceptibility of a viral strain to an anti-viral in a patient, and to anti-viral/immunosuppressive treatment regimens that also prevent graft rejection without leaving the patient vulnerable to excessive morbidity and mortality from HCV infection. There is further a need for tools which physicians can use before and during CsA or other cyclophilin inhibitor treatment to monitor development of anti-viral resistance or susceptibility by the virus, to predict and verify treatment efficacy, and to customize treatment.

### SUMMARY OF THE INVENTION

The present inventors have shown that the antiviral benefit of antiviral agents varies according to variations of the HCV genome and amino acid sequence, and that certain HCV strains display more sensitivity to antiviral agents, including cyclophilin inhibitors such as CsA, than others. Thus, the present invention provides methods and compositions for determining variation and/or mutations in genetic and/or amino acid sequences of HCV to predict the effectiveness of antiviral agents, especially cyclophilin inhibitors, in treating HCV infection in general, and in liver transplant patients in particular.

Accordingly, in one aspect, the invention encompasses a method for determining susceptibility of a hepatitis C virus (HCV) in a sample to an anti-viral agent, the method comprising determining the amino acid sequence within the HCV NS5A region and comparing said amino acid sequence to that of a reference strain. The existence of at least one variation in the viral amino acid sequence is indicative that the virus is more or less susceptible to the anti-viral agent.

In one embodiment, the at least one variation is in a consensus amino acid sequence corresponding to amino acid residues 305-328 of the wild type HCV NS5A region of SEQ ID NO:3. Because length polymorphisms occur in various HCV strains, the amino acid residue numbering of the consensus sequence can vary in different HCV strains. Preferably, the at least one mutation/variation is a proline, isoleucine, arginine, or methionine substitution at the amino acid corresponding to amino acid residue 310 of SEQ ID NO:3, wherein amino acid residue 310 is typically an alanine or threonine residue in wild-type HCV lineages. Preferably, the mutated/variant consensus sequence is selected from the group consisting of KSRRFX₁RALPV (SEQ ID NO:12), wherein X₁ is proline, isoleucine, methionine, or arginine. More preferably, the mutated/variant consensus sequence is KSRRFPRALPVWARPDYNPPLVEP.

In certain embodiments, the anti-viral agent is a cyclophilin inhibitor. Non-limiting examples of cyclophilin inhibitors for which the method could be used include Debio-025, SCY-325, and cyclosporine A (CsA). CsA is the preferred cyclophilin inhibitor for which the method is used.

In some embodiments, the sample used in the method is a clinical sample obtained from a HCV infected patient. Preferably, the patient is a liver-transplant patient.

In a second aspect, the invention encompasses an isolated polynucleotide that includes a nucleic acid sequence that encodes for a region within the HCV NS5A protein having at least one mutation/variation in a consensus amino acid sequence corresponding to amino acid residues 305-315 of the reference HCV NS5 region of SEQ ID NO:3. Preferably, the variant consensus sequence encoded by the polynucleotide is KSRRFX₁RALPVWAX₂PX₃X₄X₅PPLVEX₆, where X₁ is proline, isoleucine, arginine, or methionine; X₃, X₄, and X₅ can be any amino acid; and X₆ is proline, alanine, isoleucine, methionine, or arginine. More preferably, the mutated/variant consensus sequence encoded by the polynucleotide is KSRRFPRALPV (SEQ ID NO:13). The invention further encompasses an antiviral agent-susceptible HCV replicon that includes the isolated polynucleotide, and a gene chip including at least two such isolated polynucleotides.

In a third aspect, the invention encompasses an isolated polynucleotide that includes a nucleic acid sequence that encodes for a region within the HCV NS5A protein having at least one variation in a consensus amino acid sequence corresponding to amino acid residues 305-328 of the reference HCV NS5 region of SEQ ID NO:3, where the mutated/variant consensus sequence encoded by the polynucleotide is KSRRFX₁RALPVWAX₂PX₃X₄X₅PPLVEX₆, where X₁ is proline, isoleucine, arginine, or methionine; X₃, X₄, and X₅ can be any amino acid; and X₆ is proline, alanine, isoleucine, methionine, or arginine. Preferably, the mutated/variant consensus sequence encoded by the polynucleotide is KSRRFPRALPVWARPDYNPPLVEP The invention further encompasses an antiviral agent-susceptible HCV replicon that includes the isolated polynucleotide, and a gene chip including at least two such isolated polynucleotides.

In certain embodiments, the anti-viral agent is a cyclophilin inhibitor. Non-limiting examples of cyclophilin inhibitors for which the method could be used include Debio-025, SCY-325, and cyclosporine A (CsA). CsA is the preferred cyclophilin inhibitor for which the method is used.

In some embodiments, the sample used in the method is a clinical sample obtained from a HCV infected patient. Preferably, the patient is a liver-transplant patient.

In a fourth aspect, the invention encompasses a gene chip comprising at least two isolated polynucleotides, where at least one of the polynucleotides includes a nucleic acid sequence that encodes for a region within the HCV NS5A protein having at least one variation in a consensus amino acid sequence corresponding to amino acid residues 305-328 of the reference HCV NS5 region of SEQ ID NO:3, where the mutated/variant consensus sequence encoded by the polynucleotide is KSRRFX₁RALPVWAX₂PX₃X₄X₅PPLVEX₆, where X₁ is proline, isoleucine, arginine, or methionine; X₃, X₄, and X₅ can be any amino acid; and X₆ is proline, alanine, isoleucine, methionine, or arginine. Preferably, the kit includes at least one isolated polynucleotide, and a means for determining whether a sample contains a nucleic acid molecule that comprises the nucleotide sequence of the polynucleotide. The means of determining whether a sample contains a nucleic acid molecule that comprises the nucleotide sequence of the polynucleotide includes reagents suitable for a PCR or a hybridization reaction that utilizes the polynucleotide molecule as a primer or a probe.

In a fifth aspect, the invention encompasses a method of monitoring the development of anti-viral agent susceptibility in an HCV patient. The method includes the step of determining the amino acid sequence of a region of the NS5A protein of the HCV poly-protein in a sample from the patient. The appearance of a mutation/variant as described previously is indicative that the HCV has developed increased or decreased susceptibility to the anti-viral agent. Preferably, the patient is a liver transplant patient afflicted by HCV infection.

In a sixth aspect, the invention encompasses a method for managing HCV treatment in a patient. The method includes the steps of (1) determining whether the HCV in the patient is susceptible to a given anti-viral agent, as described previously, and (2) administering to the patient a suitable anti-viral agent or combination of agents accordingly. Preferably, the patient is a liver-transplant patient. Preferably, the one or more anti-viral agents include a cyclophilin inhibitor selected from the group consisting of Debio-025, SCY-325, and CsA.

In a seventh aspect, the invention encompasses an anti-viral agent-susceptible HCV replicon.

In an eighth aspect, the invention encompasses a method for screening for anti-viral pharmaceutical compounds. The method includes the steps of (1) applying a candidate compound to a cell culture that includes an antiviral agent-susceptible replicon as described previously, and (2) determining whether the candidate compound inhibits viral replication or viral protein synthesis. A candidate that shows inhibitory effects is an anti-viral compound.

Other objects, features and advantages of the present invention will become apparent after review of the specification, claims, and data and figures set forth herein.

### BRIEF DESCRIPTION OF THE DRAWINGS

FIG. 1 illustrates selection of four in vivo-derived mutations conferring relative CsA resistance in vitro.

FIG. 2 illustrates CsA susceptibility of naturally occurring variants in the context of an HCV 1b replicon containing the NS5A C-terminal domain derived from 1a genotype. presents data demonstrating that Con1LN-wt replicons containing different lengths of the carboxy terminus of NS5A genotype 1a have different susceptibility to CsA.

FIG. 3 provides a comparison of CsA susceptibility of Pro and Ser at amino acid position 328 of SEQ ID NO:3 with NS5A C-terminal gene sequences derived from pre- and post-transplant cases exposed to CsA.

FIG. 4 provides a comparison of CsA susceptibility of Pro at amino acid residue position 328 of SEQ ID NO:3 along with NS5A C-terminal gene sequences derived from pre- and post-transplant cases exposed to CsA.

FIG. 5 illustrates the role of HCV NS5A C-tails for CsA susceptibility and CypA binding. (A) The following replicons were constructed: Con1bLN, Con1bLN-5A1a, Con1bLN-5A2a, and Con1bLN-5A4a. The Huh7.5 cells were electroporated with in vitro synthesized RNA derived from Con1bLN-5A1a, Con1bLN-5A2a, Con1bLN-5A4a and Con1bLN-wt replicons. Equal numbers of electroporated cells were plated. The cells were either untreated (solid lines) or treated with CsA (dotted lines) for 120 hours and luciferase activity was monitored every 24 hours and presented. (B) The percent inhibition of respective replicons in (A) were calculated and presented. (C) The CypA binding capacity of NS5A regions derived from different genotypes. The 35S labeled proteins were incubated with either GST-CypA or GST-CypA55/60. The pulled down complexes were resolved by SDS-PAGE and signal was detected after autoradiography. The arrows indicate expected size of poly-protein (I=input ( 1/20th loaded); P=pull-down).

FIG. 6 presents mutational analysis of Con1bLN-5A1a chimeric replicons which revealed linear NS5A regions that alter CsA susceptibility. (A) The HCV 1b (358 isolates) and 1a (224 isolates) amino acid sequences were retrieved from the European HCV Database and subjected to amino acid homology analysis using web based program. The amino acid alignment analysis was performed on 53 amino acids N-terminal to the DYN region of HCV NS5A from 1b and 1a genotypes. The highly conserved DYN region is underlined. Site-directed mutagenesis was performed in two cluster regions, C1 and C2, in Con1bLN-wt replicon to make it similar to genotype 1a. The boxed residues were substituted in 1b replicon with genotype 1a amino acids. Amino acids of interest are numbered. The amino acids of interest are numbered. (B) The CsA susceptibility replication assay was performed on replicons Con1bLN-laCl, Con1bLN-1aC2, Con1bLN-5A1a and Con1bLN-wt replicons. (C) The percent inhibition of respective replicons in (B) were calculated and presented.

FIG. 7 presents mutational analysis of Con1bLN-wt replicon at position 310 and analysis of CsA susceptibility. (A) Logo analysis of the C2 region of 358 isolates from genotype 1b and 224 from genotype 1a of HCV NS5A. The boxed proline residue at position 310 appears to be highly conserved among most HCV 1b viruses. Sequences derived from genotype 1b and 1a are marked. (B) The CsA susceptibility of Con1bLN-P310A, Con1bLN-P310T was compared to Con1bLN-wt replicons. (C) The percent inhibition of respective replicons in (B) were calculated and presented.

FIG. 8 illustrates CypA binding analysis of a peptide containing Ala, Pro, and Thr at position 310. (A) 35S labeled proteins derived from NS5A peptide fused to the GFP coding sequences were incubated with either GST-CypA55/60 or with GST-CypA. The pulled-down complexes were resolved by SDS-PAGE and signal was detected by autoradiography. The arrow indicates expected size proteins (I=input ( 1/20th loaded); P=pull-down). (B) Alignment CypA binding region from genotype 1b and 2a. The highly conserved DYN region is underlined and amino acids of interests are numbered. In addition to P310 described in this study, the boxed residues were demonstrated previously to regulate the Alisporivir susceptibility in genotype 2a. See Grise et al., J. Virol. 86:4811-22, 2012.

### DETAILED DESCRIPTION OF THE INVENTION

### I. In General

Before the present materials and methods are described, it is understood that this invention is not limited to the particular methodology, protocols, materials, and reagents described, as these may vary. It is also to be understood that the terminology used herein is for the purpose of describing particular embodiments only, and is not intended to limit the scope of the present invention which will be limited only by the appended claims.

As used herein and in the appended claims, the singular forms “a”, “an”, and “the” include plural reference unless the context clearly dictates otherwise. As well, the terms “a” (or “an”), “one or more” and “at least one” can be used interchangeably herein. The terms “comprising”, “including”, and “having” can be used interchangeably. The term “polypeptide” and the term “protein” are used interchangeably herein.

Unless defined otherwise, all technical and scientific terms used herein have the same meanings as commonly understood by one of ordinary skill in the art to which this invention belongs. Although any methods and materials similar or equivalent to those described herein can be used in the practice or testing of the present invention, the preferred methods and materials are now described. All publications and patents specifically mentioned herein are incorporated by reference for all purposes including describing and disclosing the chemicals, cell lines, vectors, animals, instruments, statistical analysis and methodologies which are reported in the publications which might be used in connection with the invention. All references cited in this specification are to be taken as indicative of the level of skill in the art. Nothing herein is to be construed as an admission that the invention is not entitled to antedate such disclosure by virtue of prior invention.

The practice of the present invention will employ, unless otherwise indicated, conventional techniques of molecular biology, microbiology, recombinant DNA, and immunology, which are within the skill of the art. Such techniques are explained fully in the literature. See, for example, Molecular Cloning A Laboratory Manual, 2nd Ed., ed. by Sambrook, Fritsch and Maniatis (Cold Spring Harbor Laboratory Press: 1989); DNA Cloning, Volumes I and II (D. N. Glover ed., 1985); Oligonucleotide Synthesis (M. J. Gait ed., 1984); Mullis et al. U.S. Pat. No. 4,683,195; Nucleic Acid Hybridization (B. D. Hames & S. J. Higgins eds. 1984); Transcription And Translation (B. D. Hames & S. J. Higgins eds. 1984); Culture Of Animal Cells (R. I. Freshney, Alan R. Liss, Inc., 1987); Immobilized Cells And Enzymes (IRL Press, 1986); B. Perbal, A Practical Guide To Molecular Cloning (1984); the treatise, Methods In Enzymology (Academic Press, Inc., N.Y.); Gene Transfer Vectors For Mammalian Cells (J. H. Miller and M. P. Calos eds., 1987, Cold Spring Harbor Laboratory); Methods In Enzymology, Vols. 154 and 155 (Wu et al. eds.), Immunochemical Methods In Cell And Molecular Biology (Mayer and Walker, eds., Academic Press, London, 1987); and Handbook Of Experimental Immunology, Volumes I-IV (D. M. Weir and C. C. Blackwell, eds., 1986).

Unless otherwise indicated, the art-accepted standard single letter amino acid codes are used herein to identify specific amino acids and the amino acid substitutions of the present invention. In the context of the present invention, the following abbreviations for the commonly occurring nucleic acid bases are used. “A” refers to adenosine, “C” refers to cytidine, “G” refers to guanosine, “T” refers to thymidine, and “U” refers to uridine.

The term “nucleic acid” typically refers to large polynucleotides. A “polynucleotide” means a single strand or parallel and anti-parallel strands of a nucleic acid. Thus, a polynucleotide may be either a single-stranded or a double-stranded nucleic acid. A polynucleotide is not defined by length and thus includes very large nucleic acids, as well as short ones, such as an oligonucleotide The term “oligonucleotide” typically refers to short polynucleotides, generally no greater than about 50 nucleotides. It will be understood that when a nucleotide sequence is represented by a DNA sequence (i.e., A, T, G, C), this also includes an RNA sequence (i.e., A, U, G, C) in which “U” replaces “T.”

“Polynucleotide(s)” generally refers to any polyribonucleotide or polydeoxribonucleotide, which may be unmodified RNA or DNA or modified RNA or DNA. “Polynucleotide(s)” include, without limitation, single- and double-stranded DNA, DNA that is a mixture of single- and double-stranded regions or single-, double- and triple-stranded regions, single- and double-stranded RNA, and RNA that is mixture of single- and double-stranded regions, hybrid molecules comprising DNA and RNA that may be single-stranded or, more typically, double-stranded, or triple-stranded regions, or a mixture of single- and double-stranded regions. As used herein, the term “polynucleotide(s)” also includes DNAs or RNAs as described above that contain one or more modified bases. Thus, DNAs or RNAs with backbones modified for stability or for other reasons are “polynucleotide(s)” as that term is intended herein. Moreover, DNAs or RNAs comprising unusual bases, such as inosine, or modified bases, such as tritylated bases, to name just two examples, are polynucleotides as the term is used herein. It will be appreciated that a great variety of modifications have been made to DNA and RNA that serve many useful purposes known to those of skill in the art. The term “polynucleotide(s)” as it is employed herein embraces such chemically, enzymatically or metabolically modified forms of polynucleotides, as well as the chemical forms of DNA and RNA characteristic of viruses and cells, including, for example, simple and complex cells. “Polynucleotide(s)” also embraces short polynucleotides often referred to as oligonucleotide(s).

The term “isolated nucleic acid” used in the specification and claims means a nucleic acid isolated from its natural environment or prepared using synthetic methods such as those known to one of ordinary skill in the art. Complete purification is not required in either case. The nucleic acids of the invention can be isolated and purified from normally associated material in conventional ways such that in the purified preparation the nucleic acid is the predominant species in the preparation. At the very least, the degree of purification is such that the extraneous material in the preparation does not interfere with use of the nucleic acid of the invention in the manner disclosed herein. An “isolated” polynucleotide or polypeptide is one that is substantially pure of the materials with which it is associated in its native environment. By substantially free, is meant at least 50%, at least 55%, at least 60%, at least 65%, at advantageously at least 70%, at least 75%, more advantageously at least 80%, at least 85%, even more advantageously at least 90%, at least 91%, at least 92%, at least 93%, at least 94%, at least 95%, at least 96%, at least 97%, most advantageously at least 98%, at least 99%, at least 99.5%, at least 99.9% free of these materials.

Further, an isolated nucleic acid has a structure that is not identical to that of any naturally occurring nucleic acid or to that of any fragment of a naturally occurring genomic nucleic acid spanning more than three separate genes. An isolated nucleic acid also includes, without limitation, (a) a nucleic acid having a sequence of a naturally occurring genomic or extrachromosomal nucleic acid molecule but which is not flanked by the coding sequences that flank the sequence in its natural position; (b) a nucleic acid incorporated into a vector or into a prokaryote or eukaryote genome such that the resulting molecule is not identical to any naturally occurring vector or genomic DNA; (c) a separate molecule such as a cDNA, a genomic fragment, a fragment produced by polymerase chain reaction (PCR), or a restriction fragment; and (d) a recombinant nucleotide sequence that is part of a hybrid gene. Specifically excluded from this definition are nucleic acids present in mixtures of clones, e.g., as those occurring in a DNA library such as a cDNA or genomic DNA library. An isolated nucleic acid can be modified or unmodified DNA or RNA, whether fully or partially single-stranded or double-stranded or even triple-stranded.

Conventional notation is used herein to describe polynucleotide sequences: the left-hand end of a single-stranded polynucleotide sequence is the 5′-end; the left-hand direction of a double-stranded polynucleotide sequence is referred to as the 5′-direction. The direction of 5′ to 3′ addition of nucleotides to nascent RNA transcripts is referred to as the transcription direction. The DNA strand having the same sequence as an mRNA is referred to as the “coding strand.” Sequences on a DNA strand which are located 5′ to a reference point on the DNA are referred to as “upstream sequences.” Sequences on a DNA strand which are 3′ to a reference point on the DNA are referred to as “downstream sequences.”

“Primer” refers to a polynucleotide that is capable of specifically hybridizing to a designated polynucleotide template and providing a point of initiation for synthesis of a complementary polynucleotide. Such synthesis occurs when the polynucleotide primer is placed under conditions in which synthesis is induced, i.e., in the presence of nucleotides, a complementary polynucleotide template, and an agent for polymerization such as DNA polymerase.

“Probe” refers to a polynucleotide that is capable of specifically hybridizing to a designated sequence of another polynucleotide. “Probe” as used herein encompasses oligonucleotide probes. A probe may or may not provide a point of initiation for synthesis of a complementary polynucleotide. A probe specifically hybridizes to a target complementary polynucleotide, but need not reflect the exact complementary sequence of the template. In such a case, specific hybridization of the probe to the target depends on the stringency of the hybridization conditions. Probes can be labeled with, e.g., detectable moieties, such as chromogenic, radioactive or fluorescent moieties, and used as detectable agents.

The present invention is based, at least in part, on the inventors discovery that variation or mutation of the amino acid sequence in a region of the NS5A protein of the HCV genome renders an increase or decrease in the susceptibility of HCV to anti-viral cyclophilin inhibitors, including CsA. To precisely define the specific mutation/variation that increases susceptibility to CsA, a reference amino acid sequence for wild type HCV 1a (NCBI accession no. AF009606.1, SEQ ID NO:1) and wild type HCV 1b (NCBI accession no. AJ238799.1, SEQ ID NO:2) are provided herein. A standard reference NS5A amino acid sequence is a 447 amino acid region of SEQ ID NO:3 (corresponding to amino acid residues 1973-2419 of SEQ ID NO:1). The following is this portion of the sequence for HCV 1a and HCV 1b. Each of the sequences begins at amino acid residue 305 of the NS5A region (corresponding to amino acid residue 305 of SEQ ID NO:3 or amino acid residue 2277 of SEQ ID NO:1 or SEQ ID NO:2). The consensus sequence is underlined. Note that position 310 (the sixth underlined residue) in HCV 1a and HCV 1b is alanine and proline, respectively, and that position 328 (the last underlined residue) in HCV 1a and HCV 1b is threonine or serine, respectively.

HCV 1a:
(SEQ ID NO: 4)
KSRRFARALPVWARPDYNPPLVETWKKPDYEPPVVHGCPLPPP
RSPPVPPPRKKRTVVLTESTLST
HCV 1b:
(SEQ ID NO: 5)
KSRKFPRAMPIWARPDYNPPLLESWKDPDYVPPVVHGCPLPP
AKAPPIPPPRRKRTVVLSESTVSS

Substituted amino acid residue 310 is the fifth residue of a twenty-three amino acid consensus sequence that the skilled artisan would recognize as being analogous across varying HCV amino acid sequences. The consensus sequence corresponds to amino acid residues 305-328 of SEQ ID NO:3, and amino acid residues 2277-2300 of SEQ ID NO:1 and SEQ ID NO:2. As HCV is subject to frequent mutation, there can be significant variation among individual HCV sequences. The consensus sequence is represented as KSRRFX₁RALPVWAX₂PX₃X₄X₅PPLVEX₆ (SEQ ID NO:6), where WAX₂PX₃X₄X₅ typically is WARPDYN, but can vary in one of the four amino acids labeled X₃-X₅. In the mutation referred to above, X₁ is proline, isoleucine, or methionine, signaling that the strain is more cyclophilin inhibitor sensitive than strains having other amino acids at the X₁ position. Amino acid residue 310 is typically an alanine residue in wild-type HCV lineages. In some cases, therefore, a mutation that indicates increased susceptibility of HCV to anti-viral agents, particularly to cyclophilin inhibitors such as CsA, can be a single proline, isoleucine, methionine or arginine substitution at amino acid residue 2281 of SEQ ID NO:1, which corresponds to amino acid residue 310 of SEQ ID NO:3.

Accordingly, in a first aspect, the invention described herein encompasses a method for determining susceptibility of a hepatitis C virus (HCV) in a sample to an anti-viral agent, the method comprising determining the amino acid sequence within the HCV NS5A region and comparing said amino acid sequence to that of a wild-type strain, wherein the existence of at least one mutation in the viral amino acid sequence is indicative that the virus is more or less susceptible to the anti-viral agent. In an exemplary embodiment, the at least one mutation in a consensus amino acid sequence corresponding to amino acid residues 305-328 of the wild-type HCV NS5A region of SEQ ID NO:3; more preferably, the at least one mutation comprises a proline, isoleucine, methionine, or arginine substitution at the amino acid corresponding to residue 310 of SEQ ID NO:3.

Substituted amino acid residue 328 is the twenty-third residue of a twenty-three amino acid consensus sequence that the skilled artisan would recognize as being analogous across varying HCV amino acid sequences. The consensus sequence corresponds to amino acid residues 305-328 of SEQ ID NO:3, and amino acid residues 2277-2300 of SEQ ID NO:1 and SEQ ID NO:2. As HCV is subject to frequent mutation, there can be significant variation among individual HCV sequences. The consensus sequence is represented as KSRRFX₁RALPVWAX₂PX₃X₄X₅PPLVEX₆ (SEQ ID NO:6), where WAX₂PX₃X₄X₅ typically is WARPDYN, but can vary in one of the four amino acids labeled X₃-X₅. In the mutation referred to above, X₆ is proline, alanine, isoleucine, or methionine, signaling that the strain is more cyclophilin inhibitor sensitive than strains having other amino acids at the X₆ position. Where X₆ is arginine, the amino acid residue arginine at position 328 indicates that the strain is less cyclophilin inhibitor sensitive than strains having other amino acids at the X₆ position. Amino acid residue 328 is typically a threonine or serine residue in wild-type HCV lineages.

Accordingly, in another aspect, the invention described herein encompasses a method for determining susceptibility of a hepatitis C virus (HCV) in a sample to an anti-viral agent, the method comprising determining the amino acid sequence within the HCV NS5A region and comparing said amino acid sequence to that of a wild-type strain, wherein the existence of at least one mutation in the viral amino acid sequence is indicative that the virus is more or less susceptible to the anti-viral agent. In some cases, the at least one mutation comprises a proline, isoleucine, methionine, or arginine substitution at the amino acid corresponding to residue 310 of SEQ ID NO:3, and also comprises a proline, alanine, isoleucine, methionine, or arginine substitution at the amino acid corresponding to residue 328 of SEQ ID NO:3. In a preferred embodiment, the mutated consensus sequence is KSRRFPRALPVWARPDYNPPLVEP (SEQ ID NO:7).

In certain embodiments, the anti-viral agent is a cyclophilin inhibitor. Non-limiting examples cyclophilin inhibitors include Debio-025, SCY-325, and cyclosporine A (CsA). Preferably, the cyclophilin inhibitor is CsA.

Preferably, the sample is a clinical sample obtained from a HCV infected patient, including without limitation a liver-transplant patient. Clinical samples useful in the practice of the methods of the invention can be any biological sample from which any of genomic DNA, mRNA, unprocessed RNA transcripts of genomic DNA or combinations of the three can be isolated. As used herein, “unprocessed RNA” refers to RNA transcripts which have not been spliced and therefore contain at least one intron. Suitable biological samples are removed from human patient and include, but are not limited to, blood, buccal swabs, hair, bone, and tissue samples, such as skin or biopsy samples. Biological samples also include cell cultures established from an individual.

Genomic DNA, mRNA, and/or unprocessed RNA transcripts are isolated from the biological sample by conventional means known to the skilled artisan. See, for instance, Sambrook et al. (2001, Molecular Cloning: A Laboratory Manual, Cold Spring Harbor Laboratory Press, Cold Spring Harbor, N.Y.) and Ausubel et al. (eds., 1997, Current Protocols in Molecular Biology, John Wiley & Sons, New York). The isolated genomic DNA, mRNA, and/or unprocessed RNA transcripts can be used, with or without amplification, to detect a mutation relevant to the invention.

A variety of methodologies may be adapted by routine optimization to facilitate polypeptide or nucleotide sequence determination of HCV NS5A regions of interest. For example, nucleotide sequence information may be obtained by direct DNA sequencing of HCV NS5A region nucleic acid contained in a biological sample obtained from a patient of interest (e.g., a blood sample). The assay may be adapted to use a variety of automated sequencing procedures (Naeve et al., Biotechniques 19:448-453, 1995), including sequencing by mass spectrometry (see, e.g., PCT International Publication No. WO 94/16101; Cohen et al., Adv. Chromatogr. 36:127-162, 1996; and Griffin et al., Appl. Biochem. Biotechnol. 38:147-159, 1993). Traditional sequencing methods may also be used, such as dideoxy-mediated chain termination method (Sanger et al., J. Molec. Biol. 94:441, 1975; Prober et al., Science 238:336-340, 1987) and the chemical degradation method (Maxam et al., PNAS 74:560, 1977).

A preferred sequencing method for detection of a single nucleotide change is pyrosequencing. See, for instance, Ahmadian et al., Anal. Biochem, 280:103-110, 2000; Alderborn et al., Genome Res. 10:1249-1258, 2000; and Fakhrai-Rad et al., Hum. Mutat. 19:479-485, 2002. Pyrosequencing involves a cascade of four enzymatic reactions that permit the indirect luciferase-based detection of the pyrophosphate released when DNA polymerase incorporates a dNTP into a template-directed growing oligonucleotide. Each dNTP is added individually and sequentially to the same reaction mixture, and subjected to the four enzymatic reactions. Light is emitted only when a dNTP is incorporated, thus signaling which dNTP in incorporated. Unincorporated dNTPs are degraded by apyrase prior to the addition of the next dNTP. The method can detect heterozygous individuals in addition to heterozygotes. Pyrosequencing uses single stranded template, typically generated by PCR amplification of the target sequence. One of the two amplification primers is biotinylated thereby enabling streptavidin capture of the amplified duplex target. Streptavidin-coated beads are useful for this step. The captured duplex is denatured by alkaline treatment, thereby releasing the non-biotinylated strand.

In a third aspect, the invention encompasses an isolated polynucleotide comprising a nucleic acid sequence that encodes for a region within the HCV NS5A protein having at least one mutation in a consensus amino acid sequence corresponding to amino acid residues 305-328 of the wild type HCV NS5 region of SEQ ID NO:3, wherein the mutated consensus sequence encoded by the polynucleotide is KSRRFX₁RALPVWAX₂PX₃X₄X₅PPLVEX₆ (SEQ ID NO:6), where X₁ can be a proline, isoleucine, or methionine; where WAX₂PX₃X₄X₅ can be WARPDYN, but can vary in one of the four amino acids labeled X₃-X₅; and where X₆ can be proline, alanine, isoleucine, or methionine, signaling that the strain is more cyclophilin inhibitor sensitive than strains having other amino acids at the X₆ position. In some such embodiments, the mutated consensus sequence encoded by the polynucleotide is KSRRFPRALPVWARPDYNPPLVEP (SEQ ID NO:7).

Two or more such polynucleotides may be included in a diagnostic kit, microarray, or gene chip used to carry out detection methods according to the invention. The polynucleotide may also be incorporated in an antiviral agent-susceptible HCV replicon.

Amplification of a polynucleotide sequence according to the invention may be carried out by any method known to the skilled artisan. See, for instance, Kwoh et al. (Am. Biotechnol. Lab. 8:14-25, 1990) and Hagen-Mann et al. (Exp. Clin. Endocrinol. Diabetes 103:150-155, 1995). Amplification methods include, but are not limited to, polymerase chain reaction (“PCR”) including RT-PCR, strand displacement amplification (Walker et al., PNAS 89:392-396, 1992; Walker et al., Nucleic Acids Res. 20:1691-1696, 1992), strand displacement amplification using Phi29 DNA polymerase (U.S. Pat. No. 5,001,050), transcription-based amplification (Kwoh et al., PNAS 86:1173-1177, 1989), self-sustained sequence replication (“35R”) (Guatelli et al., PNAS 87:1874-1878, 1990; Mueller et al., Histochem. Cell Biol. 108:431-437, 1997), the Q.beta. replicase system (Lizardi et al., BioTechnology 6:1197-1202, 1988; Cahill et al., Clin. Chem. 37:1482-1485, 1991), nucleic acid sequence-based amplification (“NASBA”) (Lewis, Genetic Engineering News 12(9):1, 1992), the repair chain reaction (“RCR”) (Lewis, 1992, supra), and boomerang DNA amplification (or “BDA”) (Lewis, 1992, supra).

PCR may be carried out in accordance with known techniques. See, e.g., Bartlett et al., eds., 2003, PCR Protocols Second Edition, Humana Press, Totowa, N.J. and U.S. Pat. Nos. 4,683,195; 4,683,202; 4,800,159; and 4,965,188. In general, PCR involves, first, treating a nucleic acid sample (e.g., in the presence of a heat stable DNA polymerase) with a pair of amplification primers. One primer of the pair hybridizes to one strand of a target polynucleotide sequence. The second primer of the pair hybridizes to the other, complementary strand of the target polynucleotide sequence. The primers are hybridized to their target polynucleotide sequence strands under conditions such that an extension product of each primer is synthesized which is complementary to each nucleic acid strand. The extension product synthesized from each primer, when it is separated from its complement, can serve as a template for synthesis of the extension product of the other primer. After primer extension, the sample is treated to denaturing conditions to separate the primer extension products from their templates. These steps are cyclically repeated until the desired degree of amplification is obtained. The amplified target polynucleotide may be used in one of the detection assays described elsewhere herein to identify the mutation present in the amplified target polynucleotide sequence.

In a fourth aspect, the invention encompasses a kit comprising at least one isolated polynucleotide as described above and a means for determining whether a sample contains a nucleic acid molecule that comprises the nucleotide sequence of the polynucleotide. The means for determining whether a sample contains a nucleic acid molecule may include reagents suitable for a PCR or a hybridization reaction that utilizes the polynucleotide molecule as a primer or a probe.

More specifically, the kit may contain at least one pair of amplification primers that is used to amplify a target HCV NS5A nucleotide region containing one of the mutations identified in the invention. The amplification primers are designed based on the sequences provided herein for the upstream and downstream sequence flanking the mutation. In a preferred embodiment, the amplification primers will generate an amplified double-stranded target polynucleotide between about 50 base pairs to about 600 base pairs in length and, more preferably, between about 100 base pairs to about 300 base pairs in length. In another preferred embodiment, the mutation is located approximately in the middle of the amplified double-stranded target polynucleotide.

The kit may further contain a detection probe designed to hybridize to a sequence 3′ to the mutation on either strand of the amplified double-stranded target polynucleotide. In one variation, the detection probe hybridizes to the sequence immediately 3′ to the mutation on either strand of the amplified double-stranded target polynucleotide but does not include the mutation. This kit variation may be used to identify the mutation by pyrosequencing or a primer extension assay. For use in pyrosequencing, one of the amplification primers in the kit may be biotinylated and the detection probe is designed to hybridize to the biotinylated strand of the amplified double-stranded target polynucleotide. For use in a primer extension assay, the kit may optionally also contain fluorescently labeled ddNTPs. Typically, each ddNTP has a unique fluorescent label so they are readily distinguished from each other.

Any of the above kit variations may optionally contain one or more nucleic acids that serve as a positive control for the amplification primers and/or the probes. Any kit may optionally contain an instruction material for performing risk diagnosis.

In a fifth aspect, the invention encompasses a method of monitoring the development of anti-viral agent susceptibility in an HCV patient, the method comprising determining the amino acid sequence of a region of the NS5A protein of the HCV poly-protein in a sample from the patient, wherein the appearance of the mutation/variant characterized previously is indicative that the HCV has developed increased or decreased susceptibility to the anti-viral agent. Again, the method could be used with a liver transplant patient afflicted by HCV infection. Such a method could be extended to the management of HCV treatment in a liver-transplant patient. Such a method would include the steps of determining whether the HCV in the patient is susceptible to a given anti-viral agent, and administering to the patient a suitable anti-viral agent or combination of agents accordingly.

Detection of proline, leucine, arginine, or methionine at position X₁ in the consensus sequence KSRRFX₁RALPVWAX₂PX₃X₄X₅PPLVEX₆ (SEQ ID NO:6) (i.e., amino acid residue 310 of SEQ ID NO:3, and amino acid residue 2281 of SEQ ID NO:1 and SEQ ID NO:2) of HCV in a patient sample indicates that a cyclophilin inhibitor should be included in the antiviral regimen provided to that patient. In contrast, detection of alanine at position X₁ in the consensus sequence of HCV in a patient sample indicates that a cyclophilin inhibitor should not be included in the antiviral regimen provided to that patient. Similarly, detection of proline or alanine at position X₆ in the consensus sequence KSRRFX₁RALPVWAX₂PX₃X₄X₅PPLVEX₆ (SEQ ID NO:6) (i.e., amino acid residue 328 of SEQ ID NO:3, and amino acid residue 2300 of SEQ ID NO:1 and SEQ ID NO:2) of HCV in a patient sample indicates that a cyclophilin inhibitor should be included in the antiviral regimen provided to that patient. In contrast, detection of arginine at position X₆ in the consensus sequence of HCV in a patient sample indicates that a cyclophilin inhibitor should not be included in the antiviral regimen provided to that patient.

In a sixth aspect, the invention encompasses a method for screening for anti-viral pharmaceutical compounds. The method includes the steps of applying a candidate compound to a cell culture that comprises an antiviral agent-susceptible replicon as described previously, and determining whether the candidate compound inhibits viral replication or viral protein synthesis. A candidate that shows inhibitory effects is a demonstrated anti-viral compound.

The embodiments described here and in the following example are for illustrative purposes only, and various modifications or changes apparent to those skilled in the art are included within the scope of the invention. The terminology used to describe particular embodiments is not intended to limit the scope of the present invention, which is limited only by the claims. The following examples are offered to illustrate, but not to limit, the scope of the present invention.

### EXAMPLES

### Example 1

### Variation at Position 328 Correlates with HCV Susceptibility to Cyclophilin Inhibition

Introduction

HCV is one of the most common indications for liver transplant worldwide. After liver transplantation though HCV, reinfection is nearly universal, and the disease is typically more aggressive in the now immunosuppressed patient than it was pre-transplant. The optimal immunosuppression regimen for HCV infected transplant patients are unclear. Since HCV replication requires the host cofactor cyclophilin, the cyclophilin inhibitor and immunosuppressant CsA has been suggested as preferred over the more common immunosuppressant tacrolimus. Some but not all prospective studies have failed to detect a benefit from CsA. Strains of HCV may not be are equally susceptible to cyclophilin inhibition, and the selection of CsA resistant HCV post transplant could also obscure a benefit. The inventors' previous studies demonstrated that a proline residue at amino acid position 328 results in increased susceptibility to anti-viral CsA treatment. Their data also demonstrated increased susceptibility of the 1b con1 replicon to CsA treatment from its baseline. In these studies, the Con1LN-1a chimeric replicon was manipulated to contain alanine, isoleucine, methionine, or arginine residues at amino acid position 328 relative to SEQ ID NO:3. Alanine, isoleucine, methionine, and arginine are naturally present at amino acid position 328 relative to SEQ ID NO:3 within the HCV genotype 1a, but these residues are present at very low frequencies relative to threonine or serine residues at the same amino acid position. CsA susceptibility of replicons containing alanine, isoleucine, methionine, or arginine residues at amino acid position 328 of SEQ ID NO:3 was tested as described in U.S. application Ser. No. 13/229,271 (now published as U.S. Patent Publication No. 2012/0077738). It was suggested that the 328 variant would also correlate with increased susceptibility to more potent cyclophilin inhibitors such as Debio-025 and SCY325.

Materials and Methods

Cells, Media and Chemicals.

Huh7.5 cells were propagated in Advanced DMEM (Invitrogen, Cat. No. 12491023) containing 1× Glutamine (Invitrogen, Cat. No. 25030164), 1× Penicillin/Streptomycin (Invitrogen, cat. 15140122) and 1× non-essential amino acids (Invitrogen, Cat. No. 11140050). For these studies, the neomycin resistance gene in this replicon was replaced with a renilla luciferase-neo fusion gene which was amplified from another HCV replicon (Ikeda et al., Biochem. Biophys. Res. Comm. 329:1350-9, 2005) and termed Con1-Luciferase-Neomycin (Con1 LN) replicon which was used previously. Fernandes et al., PLoS One 5:e9815, 2010; Fernandes et al., Hepatology 46:1023-33, 2007. CsA was purchased from Sigma-Aldrich (St. Louis, Cat. No. C3662) and resuspended in absolute ethanol before use.

RNA Transcription and Transient Replication Assay.

Replicon DNA was linearized with XbaI (New England Biolabs) and transcribed using a MEGAscript T7 kit (Applied Biosystems, Cat. No. AMB1334) as per manufacturer's protocol. Six micrograms of purified RNA were electroporated into 2×10⁶ Huh7.5 cells using Gene Pulser Xcell electroporation system 250V, 850 uF, ∞R, 4 cm cuvette (Bio-Rad, CA). The electroporated cells were divided into two halves and seeded into twenty-four well plates. After the cells were attached the media was aspirated and replaced with fresh media for the first half, while the other half was treated with 0.5 μg/ml of CsA. The cells were further incubated and harvested from both sets at five different time points (24, 48, 72, 96, and 120 hrs) and renilla luciferase activity was monitored as per manufacturer's protocol. In brief, the cells were lysed with 100 μl of Renilla Lysis buffer supplied with the Renilla Luciferase kit (Promega, WI, cat. E2810). 5 μl of clarified cell lysate was mixed with 45 μl of Renilla Luciferase Assay buffer and read in triplicate on a Glomax 20/20 Luminometer (Promega, WI, USA). The average of three independent assays was calculated and data was analyzed.

Primers Used to Create Mutation:

The patient derived HCV genome was PCR amplified using the primers listed below. The expected size PCR product was digested with XhoI and BstZ17I restriction sites and cloned directionally into the Con1bLN replicon (previously described from our lab). Mutant replicons were tested for CsA sensitivity as described.

Forward primer:
(SEQ ID NO: 8)
5′ TTCGCTCGAGCCCTGCCCGTTTGGGCGCGGCCGGACTACAACCCC
CCGCTAGTAGAGCCCTGGAAAAAG 3′
Reverse primer:
(SEQ ID NO: 9)
5′ CCATGTATACGACATTGAGCAGCAG 3′

Genetic Manipulation of HCV Replicon:

The Con1bLN replicon was digested with XhoI and BstZ17I restriction enzymes (New England Biolabs) and a corresponding fragment from HCV genotype 1a genotype (aa 311-448; ARALPVWARP (SEQ ID NO:21) to TEDVVCC (SEQ ID NO:16), accession no. AF009606) was cloned into the replicon, termed Con1bLN-5A1a (chimera). This chimeric replicon was tested for its replication efficiency and was determined to be replication competent in a tissue culture system. The chimeric replicon was further utilized for cloning homologous fragments derived from pre- and post-liver transplant individuals infected with HCV.

Results and Discussion

Samples from nine patients treated with CsA were amplified pre- and post-transplant. Eight of nine patients were genotype 1a. The genotype 1b patient acquired mutations in NS5A at residues 320 and 328 (FIG. 1). The mutation at residue 320 was not clearly associated with CsA resistance by replicon analysis, while the selection of a proline to serine change at position 328 in patient 203-002 was associated with less replicon susceptibility to CsA.

Referring to FIG. 2, the chimeric replicon was mutated from Thr to Ala, Ile, Met, Arg, Pro, and transient replication assays were performed as described previously. Note the solid line (no CsA treated) versus dashed lines (CsA treated) replicons at a particular time. Pro is suppressed much more than the Arg or Ile, suggesting a role for amino acid position in cyclophilin susceptibility in HCV genotypes 1a and 1b.

Referring to FIG. 3, the PCR fragments spanning C-terminal domain of NS5A were amplified from pre- and post-transplant/CsA treated patients who acquired 4 mutations in that region. The fragments were cloned into an HCV replicon and replication capacity was monitored as described before. The HCV replicon was also engineered to contain Pro and Ser at 328 amino acid position and replication efficiency was compared. It was observed that the replicon carrying Pro at amino acid 328 amino along with the replicon carrying gene sequences derived from pre-transplant patient were most sensitive to CsA treatment. These data further confirm the involvement of amino acid position 328 in cyclophilin susceptibility.

As shown in FIG. 4, the HCV 1b replicon was engineered to contain Pro at 328 amino acid position and replication efficiency was compared along with pre- and post-transplant. As expected, the replicons carrying Pro at amino acid 328 along with the replicon carrying gene sequences derived from pre-transplant patient were most sensitive to CsA treatment.

### Example 2

### Subtype Specific Differences in NS5A Domain II Correlate with HCV Susceptibility to Cyclophilin Inhibition

Materials and Methods

Cells, Antibodies, Reagents:

Huh7.5 cells were maintained as described in Fernandes et al., PLoS One 5:e9815 (2010). CsA was purchased from Sigma. Western blots were performed with Protein Disulfide Isomerase antibody as s loading control and with anti-NS5A 48 hours after RNA electroporation.

Genetic Manipulation of Con1bLN Replicon:

A cloning strategy similar to that used to obtain Con1bLN-5A1a was used to clone HCV genotype 2a fragment (aa 307-466; FRRPLPAWARP (SEQ ID NO:17) to EEDDTTVCC (SEQ ID NO:18), accession no. AB047639) and HCV genotype 4a (aa 313-449; RALPIWARPDYN (SEQ ID NO:19) to VSGSEDVVCC (SEQ ID NO:20), accession no. Y11604.1), and termed Con1bLN-5A2a and Con1bLN-5A4a, respectively. An overlapping PCR strategy was adopted to incorporate mutations into Con1bLN-5A1a312-448 chimeric replicons (Con1bLN-1aC1 and Con1bLN-1aC2. Con1bLN-1aC1 comprises a mutation of amino acids EQ to DV. The following primers were used for the overlapping PCR:

Con1bLN-1aC1
Forward:
(SEQ ID NO: 21)
5′-GTAATTTTGGACTCTTTCGATCCGCTCGTGGCGGAGGAGGATGAG-3′
Reverse:
(SEQ ID NO: 22)
5′-CCATGTATACGACATTGAGCAGCAG-3′
Con1bLN-1aC2
Forward:
(SEQ ID NO: 23)
5′-GAGATCCTGCGGAAGTCCAGGAGATTCGCCCGAGCGCTGCCC
GTTTGGGCACGC-3′
Reverse:
(SEQ ID NO: 24)
5′-CCATGTATACGACATTGAGCAGCAG-3′
Con1bLN-5A2a
Forward:
(SEQ ID NO: 25)
5′ GTTTCGTCGACCCTTACCGGCTTGGGCACGGCCTG-3′
Reverse:
(SEQ ID NO: 26)
5′ CCAGGTATACGACATGGAGCAGCACACGG 3′

Generation of JFH-LN Replicon and JFH-Luc Infectious Clone and Variants:

The JFH-LN subgenomic replicon was developed and characterized by manipulating the JFH-1 clone (Wakita et al., Nat Med 11:791-6, 2005). This clone was used for generating replicons containing two different lengths of C-tail of HCV NS5A 1a genotype (from amino acids RKKRTVVLTE (SEQ ID NO:15) to TEDVVCC (SEQ ID NO:16) for 356-448 clone and from amino acids WARPDYNPP (SEQ ID NO:14) to TEDVVCC (SEQ ID NO:16) for 312 to 448 clone). The chimeric replicons were named JFH-LN 356-4481a and JFH-LN 312-4481a. The JFH-1 done was manipulated to contain Luciferase coding sequences after HCV 5′ UTR and an EMCV IRES region to express the entire HCV coding region. To generate JFH-Luc infectious clones with 1a C-tails, the DNA fragments from respective chimeric replicons was excised with SanDI and BsrGI restriction enzymes and cloned in into JFH-Luc to give rise to JFH-Luc 356-4481a and JFH-Luc 312-4481a clones. All the clones were confirmed by nucleotide sequencing and details of generation of above constructs can be provided upon requests.

RNA Transcription and Transient Replication Assay:

The RNA transcription and electroporation was performed as described before. Fernandes et al., PLoS One 5:e9815 (2010). The electroporated cells were divided into two halves and seeded into twenty-four well plates. After six hours of incubation, both halves were given fresh media, but only one was treated with 0.5 μg/ml of CsA. The cells were further incubated and renilla luciferase activity was monitored as per the manufacturer's protocol every 24 hours. In all luciferase assays, an average of three independent assays was calculated and data was presented. Error bars represent standard deviations.

Assaying Effect of CsA on Genotype 2a Infectious Clones:

Following RNA electroporation the supernatant was collected after 96 hrs, filtered through 0.45 g filter and passed onto fresh Huh7.5 cells seeded a day before in 24 wells plate. After virus adsorption the media was aspirated and fresh media was added and further incubated for another 48 hours in the presence or absence of CsA. The cells were processed to monitor the renilla luciferase activity.

Western Blot Analysis:

The RNA electroporated Huh7.5 cells were incubated for 48 hrs and processed for western blot to detect NS5A protein using anti-NS5A monoclonal antibodies. The same blot was processed for Western analysis to detect protein disulfide isomerase (PDI) to show similar protein loadings.

Logo Analysis:

A total of 358 sequences derived from genotype HCV 1b and 224 sequences from HCV 1a genotype were retrieved from The European HCV Database, available at euhcvdb.ibcp.fr/euHCVdb/ on the World Wide Web. The sequences were subjected to Logo analysis using a web-based program available at biovirus.org on the World Wide Web. The results are presented in FIG. 7A.

Cloning Genotypic Variants:

The short stretch of carboxy terminal regions of different genotypic variants of HCV NS5A were PCR amplified and cloned directionally in the Con1b-LN replicon. The forward and reverse primers used to amplify genotypes 1a and 4a were designed to contain the XhoI and BstZ17I restriction enzyme recognition sequences, respectively, whereas primers to amplify genotypes 2a contained SalI and BstZ17I restriction enzyme recognition sequences. Plasmid DNA was linearized with XbaI and used for in vitro RNA synthesis using MEGAscript T7 kit (Invitrogen). To test CsA susceptibility of Con1bLN-wt and chimeric replicons, in vitro synthesized RNA was electroporated in Huh7.5 cells and equal numbers of cells were plated in 24-well plates. The electroporated cells were treated with either 0.5 μg/mL CsA or control. The cells were lysed with 100 μL of renilla luciferase lysis buffer, and 5 μL of cleared lysate was used to evaluate luciferase activity using the Renilla Luciferase Assay system. PCR fragments corresponding to the 311-447 region in genotypes 1a, 1b, 2a and 4a of NS5A were cloned into a T7-based expression vector and labeled ΔNS5A1a, ΔNS5A1b, ΔNS5A2a, and ΔNS5A4a. All of the clones were verified by sequencing before protein expression. Cell-free translation was performed using TnT T7 Quick Coupled Transcription/Translation System (Promega, Madison, Wis., USA) in the presence of EasyTag™ EXPRESS35S Protein Labeling Mix, [³⁵S] (Perkin Elmer).

In Vitro CypA Binding Assays:

CypA binding was performed as described previously [12]. Briefly, ³⁵S-labeled polypeptides from different NS5A carboxy termini were incubated with either GST-CypA55/60 or with GST-CypA overnight at 4° C. The GST-CypA55/60 is an active site mutant version of GST-CypA in which amino acids R55 and F60 were mutated to alanine, respectively. The bound complexes were washed five times with PBS containing 0.25% NP-40 with shaking every 5 minutes at 4° C. The complexes were resolved by SDS-12% PAGE and exposed to an X-ray film. A similar CypA binding strategy was performed for peptide tagged GFP expressed proteins. Briefly, a 15-AA peptide (LRRSRKFPRAMPIWA; SEQ ID NO: 10) was genetically engineered to be N-terminally fused to GFP coding sequence. The protein was expressed as above and was used for CypA-binding analysis as described above. All the constructs described above were sequenced to confirm desired mutations.

Results and Discussion

Previously, we demonstrated a HCV NS5A::CypA interaction and mapped the NS5A region that contributes most to the CypA binding (Fernandes et al., PLoS One 5:e9815 (2010)). The differences in CsA susceptibility between 1b and 1a and the role of NS5A C-tails in CsA susceptibility and CypA binding were further investigated. We first analyzed the amino acids sequence homology between 1a and 1b genotype outside the region that we tested above but within the NS5A region that contributes most to CypA binding. We observed a limited number of differences in the H771a genotype compared to the Con1b in the region approximately 50 amino acids N-terminal to WARPDYN (SEQ ID NO:14). Since there were fewer consistent differences between 1b and 1a N-terminal to 312 compared to the carboxy-terminal, we attempted to isolate a subtype “1a susceptibility” feature N-terminal to 312 rather than the “1a relative resistance” feature.

As shown in FIG. 5A, all the replicons, exhibited similar replication kinetics in the absence of CsA, thus indicating that the replaced poly-peptide derived from genotypes 1a, 2a and 4a did not have deleterious effects on viral replication. However the same replicons displayed contrasting susceptibility upon CsA treatment. The Con1bLN-5A4a replicon was found to be most susceptible (almost 100-fold less replication, FIG. 5B) to CsA treatment among all replicons. Although the Con1bLN-5A1a replicon had slightly lower replication capacity than the Con1bLN-wt replicon, the Con1bLN-5A1a replicon displayed the least susceptibility to CsA treatment (only 10-fold less replication compared to no CsA treatment). The Con1 LN-wt and Con1LN-5A2a replicons had slightly better replication capacity than the Con1LN-5A1a and Con1 LN-5A4a replicons in the absence of CsA, and showed less inhibition to CsA treatment compared to Con1LN-5A1a replicon.

To test the interaction between NS5A genotypic carboxy terminal regions and CypA, we expressed NS5A polypeptides derived from different genotypes in a cell-free translation system in the presence of 35S cysteine/methionine and performed CypA binding assays. We observed the polypeptide derived from genotype 1a bound more efficiently than the corresponding polypeptides of 1b, 2a and 4a genotypes (FIG. 5C). The polypeptides derived from genotype 1b and 2a bound to CypA but with less efficiency compared to genotype 1a (FIG. 5C, input lanes 4 and 7; pull-down lanes 6 and 9). The apparent migration of protein derived from 2a genotype of similar length in SDS-PAGE was slower compared to 1b genotype due to the presence of an additional 23 amino acids. The genotype 4a polypeptide displayed the least CypA binding in similar experimental set up (FIG. 5C, input lane 10 with pull-down lane 12). In general, these CypA binding patterns correlated well with their respective replicons' susceptibility towards CsA (FIG. 5A). In all the pull-down assays, an active site mutant protein CypA55/60 was used as negative control. Overall, the CypA binding data indicated that the carboxy terminal regions of NS5A both interact with CypA as expected and correlate to some degree with the ability of the replicon to replicate in the presence of CsA.

Shown in FIG. 6A is the amino acid sequence homology between genotype 1a and 1b amino acids N-terminal to an extremely highly conserved region, WARPDYN (SEQ ID NO:14), but within the NS5A region that contributes most to CypA binding as observed in our previous studies. We observed two distinct clusters that have noticeable amino acids changes, named Cluster1 (C1) and Cluster2 (C2) (FIG. 6A). To explore the role of this region towards CsA susceptibility, two additional chimeric replicons were constructed, with the replicons encompassing amino acids 267-312 and the 267-448 region derived from genotype 1a, in the backbone of Con1LN-wt replicon. The susceptibility of each replicon (Con1bLN-5A267-448 and Con1bLN-5A267-312) was compared to that of Con1bLN-5A1a (hereinafter referred to as Con1bLN-5A1a312-448). These replicons replicated well in the absence of CsA but had different degrees of susceptibility to CsA (FIG. 6B). The replicon containing the 1a region from 267-312 (purple lines) was the most sensitive to CsA (˜3 log, ˜99% reduction, FIG. 6B) suggesting that the 267-3121a stretch conferred additional susceptibility to Con1bLN-wt replicon. While the Con1bLN-wt (dotted red) and the Con1bLN-5A267-448 replicon (dotted green lines) displayed similar CsA-like susceptibility to the Con1bLN-5A1a267-312 at 96 hours in FIG. 6B, notice the clear separation at an earlier time point (compare dotted purple to dotted red/green). These data suggest the relatively increased susceptibility of the Con1bLN-5A1a267-448 replicon, as compared to Con1bLN-5A1a312-448, which was due to loss of the 267-3121b region. To our knowledge, this is the first observation that variation ˜10 amino acids N-terminal to WARPDYN (SEQ ID NO:14) in genotype 1 alters cyclosporine susceptibility and is consistent with multiple prolines in this region being influenced by cyclophilin, as shown before (Grise et al., J. Virol. 86(9):4811-22, 2012; Hanoulle et al., J. Biol. Chem. 284:13589-601, 2009).

Further analysis of amino acid residues present in C2 among genotype 1a reveals that this amino acid sequence (RKSRRFARALPV; SEQ ID NO:13) is fairly conserved, with the exception of the alanine (FIG. 6A). The H771a genotype (AF009606) has alanine (bold underlined) at this particular position, compared to a proline in 1b. Logo analysis (Crooks et al., Genome Res. 14(6):1188-90, 2004) of the amino acids in C2 cluster of 1a and 1b demonstrates that while proline is well-conserved in the genotype 1b lineage, genotype 1a commonly has either an alanine or a threonine (FIG. 7A).

To examine the role of amino acid 310 in genotype 1b (FIG. 7A) in its native genotype 1b context, we mutated this amino acid to either an alanine or threonine. The resulting replicons (Con1bLN-P310A and Con1bLN-P310T) were tested for CsA susceptibility as described above. Both of the replicons carrying mutations A1a or Thr were more sensitive to CsA treatment than the Con1bLN-wt replicon, thus indicating a role for proline at position 310 in CypA regulation in genotype 1b (FIGS. 7B, 7C).

We next tested if CypA could bind to this stretch of amino acids and, if so, whether or not a proline at 310 and/or 314 altered binding. A 15 amino acid long peptide representing this region was engineered as an N-terminal fusion protein with GFP and GST-CypA binding assay was performed as above. The GFP alone did not bind to either CypA55/60 or GST-CypA in a pull-down assay (FIG. 8A, lane 2 and 3). The peptide tagged GFP carrying 310P/314P amino acids bound well (lane 6), whereas a peptide-tagged GFP containing 310A/314A amino acids exhibited little to no binding to CypA (FIG. 8A, lane 12), thus indicating that one or both of the prolines may contribute to CypA binding. We then expressed GFP tagged with peptides carrying mutations at the 310 and 314 positions to determine which prolines contribute to CsA susceptibility. The peptide-tagged GFP carrying mutations 310P/314A (FIG. 8A, lane 9) and 310A/314P (FIG. 8A, lane 15) bound to CypA to the same degree as 310P/314P. These data suggest that both of the prolines contribute to CypA binding. Furthermore, mutating the proline at position 310 to either A1a or Thr did not abolish the CypA binding completely (FIG. 8A, lanes 15 and 18), partly due to the fact that both peptides 310A/314P and 310T/314P comprised prolines at position 314. A single point mutation at position 310 to either A1a or Thr, however, rendered the 1b replicon more sensitive to CsA, thus indicating that the proline at 310 has a role in CypA binding.

Although we and others (Grise et al., J. Virol. 86(9):4811-22, 2012) have observed a contribution of 314P to CypA binding (FIG. 8B), the data presented herein indicates that AA 310 also participates in CypA binding and significantly contributes (directly or indirectly) to CsA susceptibility in tissue culture. Amino acid analysis of the C2 region also indicates that proline (P310) is highly conserved in non-genotype 1a HCV. Interestingly, this proline residue, along with two other prolines (P310 and P315) in genotype 2a, have been found to be in the direct vicinity of NS5A::CypA interaction region as determined by gel filtration, circular dichroism, and NMR spectroscopy (Hanoulle et al., J. Biol. Chem. 284:13589-601, 2009). The transient replication data demonstrate that mutation of residues in the C2 region, and, in particular, mutation of genotype 1b P310 to alanine or threonine, leads to increased susceptibility of the 1b replicon to CsA, thus indicating the region's critical involvement in CsA susceptibility. These data are consistent with the genotype 2a data on P310 (homologous to P314 in genotype 1b), as well as P342 of genotype 2a in CsA regulation (Grise et al., J. Virol. 86(9):4811-22, 2012), indicating critical roles for amino acids both N-terminal and C-terminal to the DYN motif. To our knowledge, the role of residue P306 in genotype 2a (corresponding to 310 in genotype 1b as identified in this study) in the cyclophilin inhibitor Alisporivir susceptibility has not been investigated (FIG. 8B).

The data presented here suggest that NS5A polymorphisms outside the conserved DYN sequence influence the degree of CsA susceptibility in HCV variants. The data are also consistent with multiple prolines in this region being influenced by cyclophilin. See, e.g., Hanoulle et al., J. Biol. Chem. 284:13589-601, 2009; Tang, Viruses 2:1621-34, 2010; Fernandes et al., PLoS One 5:e9815, 2010. The interaction between cyclophilin A and NS5A is more complex than just the WARPDYN (SEQ ID NO:14) site with subtype specific effects amino- and carboxy-terminal to WARPDYN (SEQ ID NO:14). Only mutations in C2 resulted in increased sensitivity to CsA. Such increased sensitivity was not observed with mutations in C1. Interestingly, C2 contains a proline residue around which the peptidyl-prolyl isomerase (PPI) activity to cyclophilins generally occurs (Tang, Viruses 2:1621-34, 2010). This region alters CsA susceptibility both for replicons and for whole viral production.

By making NS5A chimeras, we directly compared the cyclosporine susceptibility of specific NS5A sequences unconfounded by differences in other parts of the genome. Due to the diversity of each subtype, our results do not suggest that every genotype 1a HCV is less susceptible than every genotype 1b. Others have argued that cyclophilin inhibitors are “pangenotypic” and that the heterogeneity of NS5A does not correlate with cyclophilin inhibition. See, e.g., Chatterji et al., J Hepatol 53:50-6 (2010). These arguments were based on previous studies of NS5A genes from different genotypes (1b, 1a, 2a, and 2b) and the strong conservation of the WARPDYN binding site for CypA as identified by NMR. Hanoulle et al., J. Biol. Chem. 284:13589-601 (2009).

Nonimmunosuppressive cyclophilin innibititors such as alisprovir are in phase 2/3 clinical trials and, thus far, have demonstrated efficacy, including a genotype 3 patient being cured by a short duration alisprovir monotherapy. See, e.g., Vermehren and Sarrazin, Clin Microbiol Infect 17:122-34, 2011; Tang, Viruses 2:1621-34, 2010; and Patel and Heathcote, Gut 60:879, 2011. While the approval of protease inhibitors has greatly increased the possibility of curing HCV, small molecule inhibitors quickly select resistance in HCV unless given in combination with other antivirals with different mechanisms of action. We expect that clinical studies pairing of cyclophilin inhibitors with other NS5A and non-NS5A acting antivirals can benefit from study of genetic differences among HCV genotypes.

Other embodiments and uses of the invention will be apparent to those skilled in the art from consideration from the specification and practice of the invention disclosed herein. All references cited herein for any reason, including all journal citations and U.S./foreign patents and patent applications, are specifically and entirely incorporated herein by reference. It is understood that the invention is not confined to the specific reagents, formulations, reaction conditions, etc., herein illustrated and described, but embraces such modified forms thereof as come within the scope of the following claims.

We claim:

1. A method for determining susceptibility of a hepatitis C virus (HCV) in a sample to an anti-viral agent, the method comprising determining the amino acid sequence within the HCV NS5A region and comparing said amino acid sequence to that of a reference strain, wherein the existence of at least one variation in the viral amino acid sequence is indicative that the virus is more or less susceptible to the anti-viral agent.

2. The method of claim 1, wherein the at least one variation is in a consensus amino acid sequence corresponding to amino acid residues 305-328 of the wild type HCV NS5A region of SEQ ID NO:3.

3. The method of claim 2, wherein the at least one variation comprises a proline, isoleucine, arginine, or methionine substitution at the amino acid corresponding to amino acid residue 310 of SEQ ID NO:3.

4. The method of claim 3, wherein the at least one variation comprises a proline, alanine, isoleucine, methionine or arginine substitution at the amino acid corresponding to amino acid residue 328 of SEQ ID NO:3.

5. The method of claim 2, wherein the variant consensus sequence is KSRRFX₁RALPVWAX₂PX₃X₄X₅PPLVEX₆, wherein X₁ is proline, isoleucine, arginine, or methionine; X₃, X₄, and X₅ can be any amino acid; and X₆ is proline, alanine, isoleucine, methionine, or arginine.

6. The method of claim 5, wherein the variant consensus sequence is KSRRFPRALPVWARPDYNPPLVEP.

7. The method of claim 1, wherein the anti-viral agent is a cyclophilin inhibitor.

8. The method of claim 7, wherein the cyclophilin inhibitor is selected from the group consisting of Debio-025, SCY-325, and cyclosporine A (CsA).

9. The method of claim 8, wherein the cyclophilin inhibitor is CsA.

10. The method of claim 1, wherein the sample is a clinical sample obtained from a HCV infected patient.

11. The method of claim 10, wherein the HCV infected patient is a liver-transplant patient.

12. An isolated polynucleotide comprising a nucleic acid sequence that encodes for a region within the HCV NS5A protein having at least one variation in a consensus amino acid sequence corresponding to amino acid residues 305-328 of the reference HCV NS5 region of SEQ ID NO:3, wherein the mutated/variant consensus sequence encoded by the polynucleotide is KSRRFX₁RALPVWAX₂PX₃X₄X₅PPLVEX₆, wherein X₁ is proline, isoleucine, arginine, or methionine; X₃, X₄, and X₅ can be any amino acid; and X₆ is proline, alanine, isoleucine, methionine, or arginine.

13. The isolated polynucleotide of claim 12, wherein the variant consensus sequence encoded by the polynucleotide is KSRRFPRALPVWARPDYNPPLVEP.

14. A gene chip comprising at least two isolated polynucleotides according to claim 12.

15. A kit comprising at least one isolated polynucleotide of claim 12, and a means for determining whether a sample contains a nucleic acid molecule that comprises the nucleotide sequence of the polynucleotide.

16. The kit of claim 15, wherein the means comprises reagents suitable for a PCR or a hybridization reaction that utilizes the polynucleotide molecule as a primer or a probe.

17. A method for treating a HCV infection in an individual, the method comprising determining whether the HCV infection is susceptible to an anti-viral agent, and administering to the individual one or more anti-viral agents selected on the basis of the susceptibility determination, whereby the HCV infection is treated.

18. The method of claim 17, wherein the individual is a liver transplant patient.

19. The method of claim 17, wherein the one or more anti-viral agents comprise a cyclophilin inhibitor.

20. An anti-viral agent-susceptible HCV replicon, comprising the isolated polynucleotide of claim 12.
